(19) United States
(12) Patent Application Publication
(10) Pub. No.: US 2019/0292555 A1
(43) Pub. Date: Sep. 26, 2019

(54) PRODUCING RECOMBINANT PRODUCTS IN CONTAINED SYSTEMS

(71) Applicant: Impossible Foods Inc., Redwood City, CA (US)

(72) Inventors: Simon Christopher Davis, San Francisco, CA (US); Patrick O'Reilly Brown, Stanford, CA (US); Michael Lassner, Portland, OR (US)

(21) Appl. No.: 16/466,003

(22) PCT Filed: Dec. 1, 2017
(86) PCT No.: PCT/US2017/064160

§ 371 Date: May 31, 2019

Related U.S. Application Data
(60) Provisional application No. 62/429,557, filed on Dec. 2, 2016.

(51) Int. Cl. C12N 15/82

(52) U.S. Cl. CPC C12N 15/8238; C12N 15/8234; C12N 15/8227; C12N 15/8258

(57) ABSTRACT

This document relates to methods of making recombinant target products in transgenic plants in a contained system, and more particularly to separating the production of plant biomass from the production of the target product by selectively expressing the target product during germination in a contained system.

### CROSS-REFERENCE TO RELATED APPLICATIONS

This application claims the benefit of U.S. Application Ser. No. 62/429,557, filed Dec. 2, 2016. The disclosure of the prior application is considered part of and is incorporated by reference in the disclosure of this application.

### TECHNICAL FIELD

This disclosure relates to methods of recombinantly producing target products in transgenic plants in a contained system, such as a malting system or a hydroponic system, and more particularly, to temporally separating the production of plant biomass from the production of the target product by selectively expressing the target product during germination of transgenic seeds in the contained system and isolating the target product from the resulting seedlings.

### BACKGROUND

Transgenic plants have been developed for a variety of reasons including disease resistance, herbicide resistance, pest resistance, abiotic stress resistance (e.g., drought or salt tolerance), improved nutrition, and protein production. However, as more and more transgenic plants are developed and introduced into the environment, concerns have been raised about transfer of the transgenic DNA to conventional crops or other non-crop plants and propagation of the trait (e.g., herbicide resistance) throughout such plant populations.

### SUMMARY

This document is based on methods of using transgenic plants for recombinantly producing target products in contained systems. As described herein, in some embodiments, a recombinant nucleic acid is selectively expressed during germination (e.g., using an inducible promoter and/or germination specific promoter) and has no effect upon the plant during normal growth, avoiding possible deleterious effects of the transgene during growth of the plants and removing any selective advantage to horizontal transfer of the DNA. Seeds can be harvested from the transgenic plant and then germinated in a contained system such as a malting system or a hydroponic system, and the target product can be isolated from the resulting seedlings or plants.

Provided herein are methods of recombinantly producing a target product in a contained system. The method includes germinating a plurality of transgenic seeds in the contained system to produce a plurality of seedlings or plants, wherein each transgenic seed comprises a nucleic acid construct, contacting the seedlings or plants with an inducer for the inducible promoter; and isolating the target product from the plurality of seedlings or plants. The nucleic acid construct comprising an inducible promoter operably linked to (i) a nucleic acid encoding the target product or (ii) a nucleic acid encoding a polypeptide in a biosynthesis pathway of the target product;

Provided herein are methods of recombinantly producing a target product in a contained system. The method includes germinating a plurality of transgenic seeds in the contained system in the presence of an inducer to produce a plurality of seedlings or plants, wherein each transgenic seed includes a nucleic acid construct; and isolating the target product from the plurality of seedlings. The nucleic acid construct includes an inducible promoter operably linked to (i) a nucleic acid encoding the target product or (ii) a nucleic acid encoding a polypeptide in a biosynthesis pathway of the target product

Also provided herein are methods of recombinantly producing a target product in a contained system. The method includes germinating a plurality of transgenic seeds in the contained system to produce a plurality of seedlings or plants, wherein each transgenic seed comprises a nucleic acid construct; and isolating the target product from the plurality of seedlings. The nucleic acid construct includes a germination specific promoter operably linked to (i) a nucleic acid encoding the target product or (ii) a nucleic acid encoding a polypeptide in a biosynthesis pathway of the target product.

In any of the methods, the transgenic seeds can be soybean seeds or rice seeds. In any of the methods, the transgenic seeds can be selected from the group consisting of a beet, a sugar beet, parsnip, a bean such as an adzuki, a mung, a pea, a peanut, a lentil, or a garbanzo, a leafy vegetable such as an alfalfa, an arugula, a mustard, or a Brassica, and a grass seed such as a barley, a wheat, a corn, a rye, an oat, triticale, or spelt.

In some embodiments, the nucleic acid construct further encodes a targeting sequence. In some embodiments, the targeting sequence is a root targeting sequence. In some embodiments, the targeting sequence is a hypocotyl targeting sequence. In some embodiments, the targeting sequence is a vacuole targeting sequence.

In any of the methods described herein, the isolating step can include processing the plurality of seedlings or plants and purifying the target product from the processed seedlings or plants.

In any of the methods described herein, the inducer can be tetracycline, dexamethasone, copper, an insecticide, an estrogen, salicylic acid, methyl jasmonate, ethanol, ethylene, or acetaldehyde. For example, the inducer can be ethanol or acetaldehyde.

In some embodiments, the target product is expressed throughout the seedling or plant.

In some embodiments, the target product is a protein. In any of the methods described herein, the target product can be at least 0.01% of the total protein (e.g., total soluble protein) from the transgenic seedling or plant.

For example, the protein can be a therapeutic protein or a food protein. The therapeutic protein can be an antibody such as a chimeric antibody, humanized antibody, or a single chain antibody. The therapeutic protein can be a hormone such as erythropoietin, a cytokine, a growth factor, a blood factor, or a bone morphogenetic protein. The therapeutic protein can be collagen.

The target product can be an enzyme such as an industrial enzyme. In some embodiments, the industrial enzyme is a cellulase, a xylanase, a ligninase, a protease, a lipase, an amylase, an amyloglucosidease, a glucoamylase, a lactase, a peroxidase, a glucanase, a glucuronidase, a phytase, a catalase, or a laccase.

The target product can be a casein, a whey protein, a lactoferrin, a transglutaminase, a dehydrin, an oleosin, an albumin, a gluten, a glycinin, a conglycinin, a legumin, a vicilin, a conalbumin, a gliadin, a glutelin, a glutenin, a hordein, a prolamin, a phaseolin, a proteinoplast, a secalin, a triticeae gluten, a zein, a caloleosin, or a steroleosin protein.

The target product can be a member of the globin family. The target product can be leghemoglobin (lbc2), a non-symbiotic hemoglobin, an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin, a truncated 2/2 globin, a hemoglobin 3, a cytochrome, or a peroxidase.

The target product can be a virus or a small molecule. In some embodiments, the small molecule is a pharmaceutical small molecule or an industrial small molecule. The secondary metabolite can be from a plant biosynthesis pathway (e.g., a secondary metabolite that is endogenous to the transgenic plant or heterologous to the transgenic plant). For example, the heterologous secondary metabolite can be artemisinin, Paclitaxel, podophyllotoxin, or vinblastine.

The contained system can be a hydroponic system or a malting system. The plurality of seeds can be germinated by (i) steeping the plurality of transgenic seeds in the presence of the inducer and water until the moisture content of the plurality of transgenic seeds ranges from about 30% to about 50% and (ii) ventilating the steeped plurality of seeds at a temperature from about 15° C. to about 25° C.; and the target product can be isolated from the ventilated seedling. The plurality of seeds can be germinated by (i) steeping the plurality of transgenic seeds in the presence of water and optional inducer until the moisture content of the plurality of transgenic seeds ranges from about 30% to about 50% and (ii) ventilating the steeped plurality of seeds at a temperature from about 15° C. to about 25° C. in the presence of the inducer; and the target product can be isolated from the ventilated seedling. In some embodiments, the target product is thermostable and the plurality of seedlings can be kilned before isolation. The plant can be from 1 to 50 days old when contacted with the inducer. The target product can be isolated from the plant 6 to 100 hours after induction.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DESCRIPTION OF DRAWINGS

FIG. 1 is a schematic of vector pPTN1138IF.

FIG. 2 is the nucleic acid sequence encoding Leghemoglobin Lbc2 Glyma20g33290.1 (SEQ ID NO: 1), the nucleic acid sequence encoding the vacuole targeting signal sequence of conglycinin (conglycinin signal peptide) (SEQ ID NO: 2), the nucleic acid sequence of the AlcR from A. nidulans (SEQ ID NO: 3), and the nucleic acid sequence of the AlcA promoter from A. nidulans (SEQ ID NO: 4).

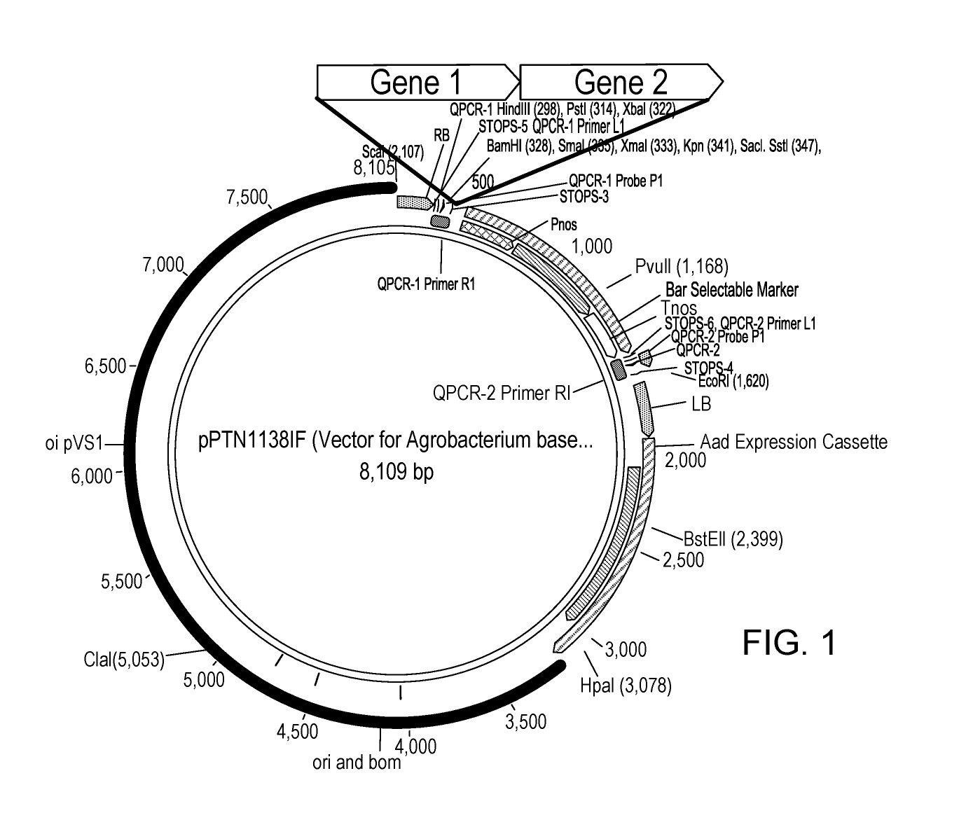

### DETAILED DESCRIPTION

In general, this document provides methods and materials for using transgenic plants to recombinantly produce target products in contained systems. As used herein, a “contained system” refers to a system for germinating a plurality of transgenic seeds (e.g., germinating the seeds in bulk such as at least 1,000 seeds, 5,000 seeds, 10,000 seeds, 50,000 seeds, 100,000 seeds, 1×10⁶ seeds, 1×10⁷ seeds, 1×10⁸ seeds, 1×10⁹ seeds, 1×10¹⁰ seeds, 1×10¹¹ seeds, 1×10¹² seeds, 1×10¹³ seeds, 1×10¹⁴ seeds, 1×10¹⁵ seeds or more) in a controlled environment (e.g., controlled temperature and humidity) such as a malting or hydroponic system. In some embodiments, 50 kg, 75 kg, 100 kg, 500 kg, 1000 kg, 10,000 kg, 25,000 kg, 50,000 kg, or 100,000 kg seeds or more are germinated. An inducer can be added at any point during or after the germination process and the target product can be isolated from the resulting seedlings or plants. For example, the target product can be isolated from the seedlings or plants 6 to 120 hours (e.g., 6 to 100, 8 to 80, 10 to 50, 10 to 20, 12 to 72, 12 to 48, or 12 to 36 hours) after induction. As used herein, the term “seedling” includes sprouted seed as well as young plants containing the hypocotyl (embryonic shoot) and the cotyledon(s), and young grains less than 6 inches tall. Germinating seeds in a greenhouse or under field conditions is not considered to be a contained system.

Malting systems are commercially available from, for example, Integrated Process Engineers & Constructors, Inc. (IPEC), Fort Atkinson, Wis., and Kaspar Schulz, Bamberg, Germany. Typically, malting refers to germinating grain to set in motion the transformation undergone naturally by the plant during its growth, and then halting that transformation depending on the desired characteristics. To prepare for germination, the transgenic seeds can be steeped with water to increase the moisture content of the seeds to about 30% to about 50% (e.g., 35% to about 50%, 40% to 50%, 42% to 48%, 43% to 47%, or 44% to 46%). Temperature is typically maintained from 15° C. to 30° C.

For example, in some embodiments, the transgenic seeds can be immersed in water and an optional inducer (e.g., ethylene gas, ethanol or acetaldehyde, e.g., to a concentration up to 2%), alternating with drainage and periods of exposure to air, until the moisture content of the seeds is about 30% to about 50%. During the immersion phase, the seeds can be turned and oxygenated using compressed air. During the drainage phase, the air can be renewed frequently to evacuate the CO₂ and heat it produces and to provide the seeds with needed oxygen.

For example, in some embodiments, the transgenic seeds can be steeped by aspersion, in which the seeds are frequently sprayed with water and optional inducer (e.g., ethylene gas, ethanol or acetaldehyde, e.g., to a concentration up to 2%), with ample renewal of the air. In either case, at the end of the steeping process, the germ and rootlets (i.e., developing roots) can appear.

The steeped transgenic seeds can be ventilated (e.g., by spreading out the seeds on a perforated substrate such as a perforated grain floor) under controlled temperature (e.g., from about 15° C. to about 25° C.) and humidity, to allow the seeds to respire and an inducer such as ethylene gas or ethanol (up to a concentration of 2%) can be added to induce expression of the target product. After three to six days, during which the seed can be regularly turned and sometimes sprayed (or “watered”), the gemmule becomes as large as the seed itself and the rootlets that have developed look withered. This stage can be referred to as green malt. The target product can be isolated from the seedlings as described below.

In some embodiments, the seedlings can be kilned by drying the seedlings at a temperature of 45 to 90° C. to a moisture content of about 3% to about 15% (e.g., 4% to 10%, 4% to 8%, 4% to 5%, 4.5% to 5.5%, 5% to 6%, 5.5% to 6.5%, 6% to 7%, 6.5% to 7.5%, 7% to 8%, 7.5% to 8.5%, 8% to 9%, 8.5% to 9.5%, 9% to 10%, or 9.5% to 10.5%). The target product can be isolated from the dried seedlings as described below.

In some embodiments, the transgenic seeds are steeped in water and ventilated as in the malting process, then once roots appear, the seeds can be placed on moistened soil, coconut coir, vermiculite or other medium, and kept covered to maintain moisture and temperature levels and keep light out. An inducer such as ethylene gas or ethanol can be added (e.g., up to a concentration of 2%) to the seedlings (e.g., when the seedlings are 0.25″ to 2″ tall) and induction of the target product can be maintained for six to 72 hours, and then the seedlings can be harvested.

In some embodiments, a hydroponics system, such as one used to produce fodder for farm animals, is used to germinate the plurality of transgenic seeds. Hydroponics is a method of agriculture that grows plants without soil using a mixture of water and nutrient salts. For example, the hydroponics system can be an ebb and flow system, a top feed system, an aeroponics system, a wicks system, or a nutrient film system. See, for example, Naik et al., Indian J. Anim. Nutr. 32 (1): 1-9 (2015), and Haddadi, Agricultural Advances 5(3) 269-274 (2016).

In some embodiments, the transgenic seeds are grown beyond the seedling stage before addition of the inducer. The plants can be grown to any stage, including maturity, before adding inducer. For example, in some embodiments, inducer can be added when the plants are from 1 to 50 days old and the target product can be isolated from the plants, for example, 6 to 100 hours after induction. The maturity of the plant at induction will be dependent upon the plant and target product being produced.

To isolate the target product from the seedlings or plants, the seedlings or plants can be removed from the contained system (e.g., a malting chamber or hydroponic growth chamber) and processed using any appropriate measure including, for example, grinders, hammer mills, shredders, chippers, screwpress, or high pressure homogenization. If the material is kilned, the dried material can be hydrated before processing.

Target proteins can be separated on the basis of their molecular weight, for example, by size exclusion chromatography, ultrafiltration through membranes, or density centrifugation. In some embodiments, target proteins can be separated based on their surface charge, for example, by isoelectric precipitation, anion exchange chromatography, or cation exchange chromatography. Target proteins also can be separated on the basis of their solubility, for example, by ammonium sulfate precipitation, isoelectric precipitation, surfactants, detergents or solvent extraction. Target proteins also can be separated by their affinity to another molecule, using, for example, hydrophobic interaction chromatography, reactive dyes, or hydroxyapatite.

In some embodiments, target proteins can be extracted in native form as described in WO 2016/054375. For example, a target protein can be extracted from the processed seedlings or plants with an aqueous solution containing polyethylene glycol (PEG) (e.g., PEG having a MW of 8000) and, optionally, a flocculant such as an alkylamine epichlorohydrin, to generate an extraction slurry that contains bulk solids and an extract; optionally adjusting the pH of the extraction slurry to a pH of 2 to 10; collecting the extract and adding salt such as magnesium sulfate to form a two-phase mixture, separating the two-phase mixture using, for example, gravity settling or centrifugation (e.g., using a disk stack centrifuge) to generate a PEG phase and a product phase; and collecting and filtering (e.g., microfiltering) the product phase to generate a filtered product phase that contains the protein. The filtered product phase can be concentrated and diafiltered to generate a target product concentrate. A product concentrate can be sterilized, e.g., by UV irradiation, pasteurization, or microfiltration, and dried, e.g., by spray drying or a freeze drying under mild conditions.

Other target products such as small molecules can be isolated from the seedlings using extraction (e.g., Soxhlet extraction, ultrasound-assisted extraction), precipitation (e.g., solvent precipitation), fractionation, hydrodistillation, and separation (e.g., HPLC) techniques. See, for example, Azmir, et al., J. Food Engineering, 117:426-436 (2013). For example, small molecules can be extracted in buffers, solvents like hexane, chloroform, methanol, acetone, combinations of solvents such as chloroform-methanol, or hexane-isopropanol, or using supercritical fluids (SCF) such as supercritical CO₂. Supercritical fluids act like liquid solvents with selective dissolving powers. Supercritical CO₂ is non-flammable, non-toxic and relatively inert. See, for example, Cuellar-Bermudez, et al. Microbial Biotechnology, 190-209 (2015).

### Recombinant Nucleic Acid Constructs

As described herein, the transgenic seeds contain a recombinant nucleic acid that can be selectively expressed such as during germination (e.g., using an inducible promoter or a germination specific promoter). The nucleic acid construct can include an inducible promoter operably linked to a nucleic acid encoding the target product or a nucleic acid encoding a polypeptide in a biosynthesis pathway of a target product.

An inducible promoter can include, for example, a core or basal promoter sequence and one or more elements such as transcriptional activator binding sites or other regulatory element to allow control of transcription. A core promoter refers to the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

For example, an inducible promoter can be a modified cauliflower mosaic virus (CaMV) 35S promoter that is responsive to tetracycline. See, Gatz, et al., Plant J. 2, 397-404 (1992), and Weinmann, et al., Plant J., 5, 559-569 (1994). For example, an inducible promoter can be dexamethasone-inducible, or dexamethasone-inducible and tetracycline-inactivatable. See, Aoyama and Chua, Plant J., 11, 605-612 (1997), Craft, et al., Plant J., 41, 899-918 (2005); Samalova, et al., Plant J., 41, 919-935 (2005); Bohner, et al., Plant J., 19, 87-95 (1999); and Bohner, S. and Gatz, Mol. Gen. Genet. 264, 860-870 (2001). For example, an inducible promoter can be responsive to copper. See, Mett, et al., Proc. Natl Acad. Sci. USA, 90, 4567-4571 (1993). For example, an inducible promoter can be responsive to an insecticide (e.g., tebufenozide or methoxyfenozide). See, Koo, et al., Plant J., 37, 439-448 (2004); Martinez, et al., Plant J., 5, 559-569 (1999); and Padidam, et al., Transgenic Res., 12, 101-109 (2003). For example, an inducible promoter can be estrogen responsive (e.g., 17 beta estradiol). See, Bruce, et al., (2000) Plant Cell, 12, 65-80 (2000); and Zuo, et al., Plant J., 24, 265-273 (2000).

For example, an inducible promoter can be responsive to salicylic acid, ethylene, or jasmonic acid. See, for example, Liu, et al., Plant Biotech. J., 11, 43-52 (2013) and Liu, et al., BMC Biotechnol., 11, 108 (2011). Salicylic acid (SA) interacts with either the Arabidopsis PR1 promoter or SA-responsive elements (SARE), which drives the expression of the nucleic acid of interest. Ethylene (ET) interacts with ethylene responsive element (ERE), which drives the expression of the nucleic acid of interest. Methyl jasmonate interacts with jasmonic acid responsive element (JAR) which drives the expression of the nucleic acid of interest. In some embodiments, the inducer (e.g., ethylene gas) can be added to the malting chamber to induce expression of the nucleic acid.

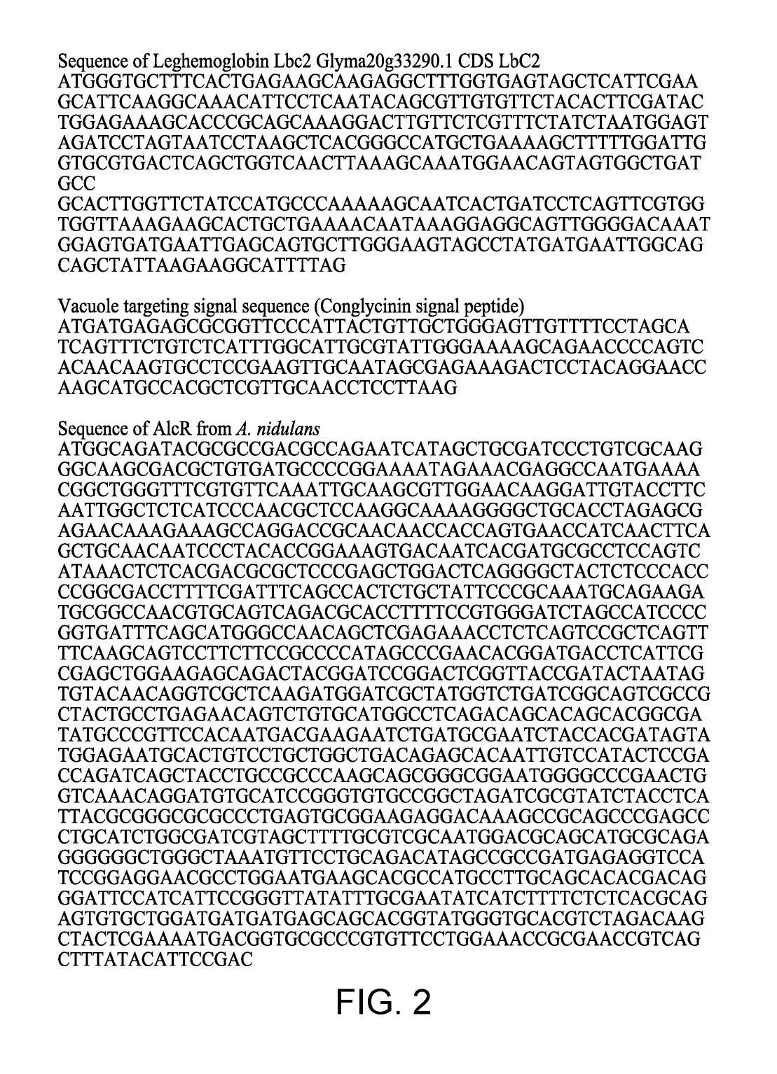

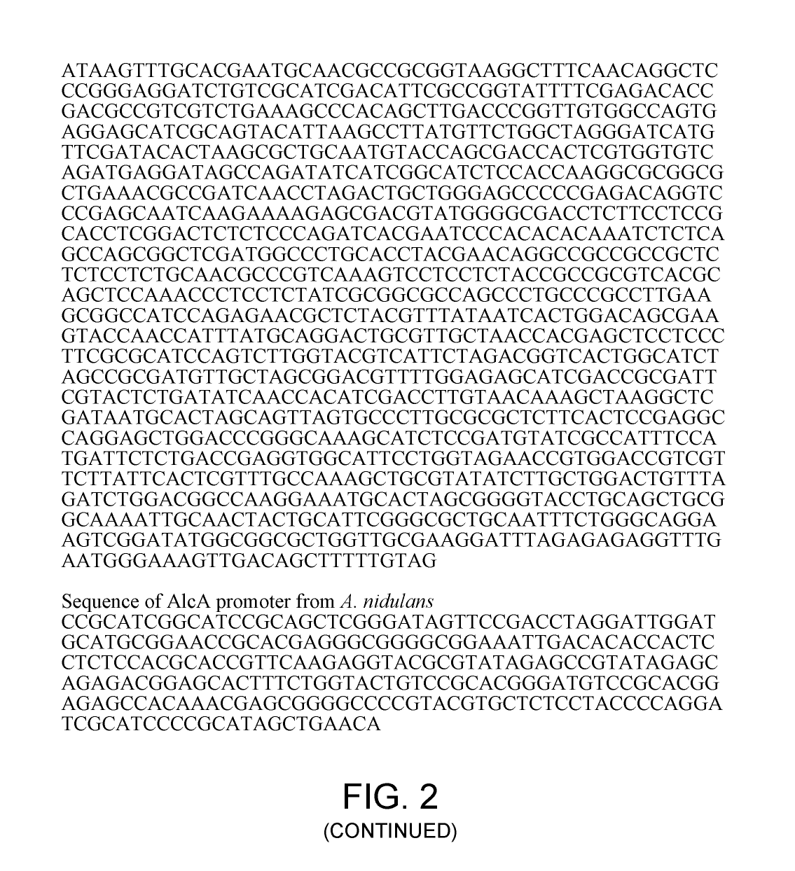

For example, an inducible promoter can be ethanol responsive (e.g., ethanol or acetaldehyde). See, Caddick, et al., Nat. Biotechnol., 16, 177-180 (1998); Roslan, et al., Plant J., 28, 225-235 (2001); and Salter, et al., Plant J., 16, 127-132 (1998). In the presence of ethanol or acetaldehyde, the Aspergillus nidulans ALCR transcription factor (alcR, see FIG. 2) drives expression from the palcA promoter by binding to upstream sequences (alcA, see FIG. 2) from the A. nidulans alcA locus. The palcA promoter is positioned upstream of a target DNA for expression.

In some embodiments, ethanol-inducible expression can be based on inducible release of viral RNA replicons from stably integrated DNA proreplicons. See, Werner, et al., Proc Natl Acad Sci USA, 108(34): 14061-14066 (2011).

A nucleic acid construct can include a germination specific promoter operably linked to a nucleic acid encoding the target product or a nucleic acid encoding a polypeptide in a biosynthesis pathway of a target product. Such a promoter results of expression of the target product during germination and/or early seedling growth in one or more of the radical, hypocotyl, cotyledons, epicotyl, root tip, shoot tip, meristematic cells, seed coat, endosperm, true leaves, internodal tissue, and nodal tissue. See, for example, promoters from genes encoding the glyoxysomal enzymes isocitrate lyase (ICL) and malate synthase (MS) from several plant species (Zhang et al., Plant Physiol. 104: 857-864, 1994); Reynolds and Smith, Plant Mol. Biol. 27: 487-497, 1995); Comai et al., Plant Physiol. 98: 53-61, 1992). Promoters also can be from other genes whose mRNAs appear to accumulate specifically during the germination process, for example class I β-1,3-glucanase B from tobacco (Vogeli-Lange et al., Plant J., 5: 273-278, 1994); canola cDNAs CA25, CA8, AX92 (Harada et al., Mol. Gen. Genet., 212: 466-473, 1988); Dietrich et al., J. Plant Nutr., 8: 1061-1073, 1992), lipid transfer protein (Sossountzove et al, Plant Cell, 3: 923-933, 1991); or rice serine carboxypeptidases (Washio et al., Plant Phys., 105: 1275-1280, 1994); and repetitive proline rich cell wall protein genes (Dana et al., Plant Mol. Biol. 14: 285-286, 1990). See U.S. Patent Publication No. 20160024512. The α-amylase promoter also can be used a germination specific promoter. See, Eskelin, et al., Plant Biotechnology Journal, 7: 657-672 (2009).

In some embodiments, the nucleic acid construct further includes a targeting sequence that can be used to direct the target product to one of several different intracellular compartments, including, for example, the endoplasmic reticulum (ER), mitochondria, plastids (such as chloroplasts), the vacuole, the Golgi apparatus, protein storage vesicles (PSV) and, in general, membranes, to structures such as the roots, or cells in, for example, the hypocotyl. Some signal peptide sequences are conserved, such as the Asn-Pro-Ile-Arg (SEQ ID NO: 5) amino acid motif found in the N-terminal propeptide signal that targets proteins to the vacuole (Marty, Plant Cell, 11: 587-599, 1999). Other signal peptides do not have a consensus sequence per se, but are largely composed of hydrophobic amino acids, such as those signal peptides targeting proteins to the ER (Vitale and Denecke, Plant Cell, 11: 615-628, 1999). Still others do not appear to contain either a consensus sequence or an identified common secondary sequence, for instance the chloroplast stromal targeting signal peptides (Keegstra and Cline, Plant Cell, 11: 557-570, 1999). Chloroplast targeting peptides commonly have a high content of hydroxylated amino acid residues (Ser, Thr, and Pro), lack acidic amino acid residues (Asp and Glu), and tend to form α-helical structures in hydrophobic environments (see, e.g., Shen, et al., Scientific Reports, 7, 46231, 2017). In some embodiments, a chloroplast targeting sequence can be a pea, rice, tobacco, Arabidopsis, or soy rubisco small subunit (rbcS) transit peptide (Van den Broeck, et al., Nature, 313, 358-363, 1985). In some embodiments, a portion of the N-terminus of the rbcS protein can be included in the targeting sequence. For example, a portion of the N-terminal unfolded region (e.g., 18, 19, 20, 21, 22, 23, 24, or 25 amino acids) of the rbcS protein can be included in the chloroplast target sequence (see, e.g., Shen, et al., 2017, supra), for a total length of 50-80 amino acids (e.g., 60, 65, 70, 75 amino acids). Furthermore, some targeting peptides are bipartite, directing proteins first to an organelle and then to a membrane within the organelle (e.g. within the thylakoid lumen of the chloroplast; see Keegstra and Cline, 1999, supra). In addition to the diversity in sequence and secondary structure, placement of the signal peptide is also varied. Proteins destined for the vacuole, for example, can have targeting signal peptides found at the N-terminus, at the C-terminus and at a surface location in mature, folded proteins.

In some embodiments, a nucleic acid construct includes a root targeting sequence such as domain A of the CaMV 35S promoter (e.g., containing a tandem repeat of the sequence TGACG separated by 7 base pairs). See, for example, Benfey, et al., The EMBO Journal, 8(8):2195-2202, 1989.

The recombinant nucleic acid is exogenous to the plant. As used herein, the term “exogenous” with respect to a nucleic acid indicates that the nucleic acid is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. A heterologous polypeptide as used herein refers to a polypeptide that is not a naturally occurring polypeptide in a plant cell, e.g., a transgenic soybean plant transformed with and expressing the coding sequence for a leghemoglobin from an alfalfa plant. “Polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

An exogenous nucleic acid also can be a sequence that is native to a plant and that has been reintroduced into cells of that plant. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

“Isolated nucleic acid” as used herein includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

“Nucleic acid” and “polynucleotide” are used interchangeably herein, and refer to both RNA and DNA, including cDNA, genomic DNA, synthetic DNA, and DNA or RNA containing nucleic acid analogs. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

The nucleic acid can be encode a target product or a nucleic acid encoding a polypeptide in a biosynthesis pathway of a target product (e.g., it can encode a polypeptide in a biosynthesis pathway that leads to the production of the target product). In some embodiments, the nucleic acid can encode multiple steps within a biosynthetic pathway or the complete biosynthetic pathway of a target product.

The target product can be a polypeptide such as a therapeutic protein or a food protein. For example, the therapeutic protein can be an antibody (e.g., a chimeric antibody, humanized antibody, or a single chain antibody), a hormone such as growth hormone, somatotropin, insulin, or erythropoietin, a cytokine such as an interleukin, an interferon, a tumor necrosis factor, a growth factor, a blood factor, a bone morphogenetic protein, collagen, or a therapeutic enzyme such as a glucocerebrosidase (for Gaucher's disease type 1), prolactazyme (for lactose intolerance), beta-lactamase (for penicillin allergy), streptokinase (for blood clots), asparaginase (for acute childhood leukemia), collagenase (for skin ulcers), DNAse (for Cystic Fibrosis), uricase/urate oxidase (for gout), glutaminase (for leukemia), hyaluronidase (for heart attack), or lysozyme (for antibiotics), rhodanase (for cyanide poisoning), trypsin (for inflammation), or urokinase (for blood clots). See Vellard, Current Opinion in Biotechnology 14:444-450, 2003

For example, the target product can be a casein, a whey protein, a lactoferrin, a transglutaminase, a dehydrin, an oleosin, an albumin, a gluten, a glycinin, a conglycinin, a legumin, a vicilin, a conalbumin, a gliadin, a glutelin, a glutenin, a hordein, a prolamin, a phaseolin, a proteinoplast, a secalin, a triticeae gluten, a zein, a caloleosin, or a steroleosin protein.

For example, the target product can be an enzyme such as industrial enzyme (e.g., a cellulase, a xylanase, a ligninase, a protease, a lipase, an amylase, an amyloglucosidease, a glucoamylase, a lactase, a peroxidase, a glucanase, a glucuronidase, a phytase, a catalase, or a laccase).

For example, the target product can be a member of the globin family (Pfam 00042). Globin family members are heme-containing proteins involved in binding and/or transporting oxygen. The terms “heme cofactor” and “heme” are used interchangeably and refer to a prosthetic group bound to iron (Fe2+ or Fe3+) in the center of a porphyrin ring. The term “heme containing protein” can be used interchangeably with “heme containing polypeptide” or “heme protein” or “heme polypeptide” and includes any polypeptide that can covalently or noncovalently bind a heme moiety. In some embodiments, the heme-containing polypeptide is a globin and can include a globin fold, which comprises a series of seven to nine alpha helices. Globin type proteins can be of any class (e.g., class I, class II, or class III), and in some embodiments, can transport or store oxygen. For example, a heme-containing protein can be a non-symbiotic type of hemoglobin or a leghemoglobin. A heme-containing polypeptide can be a monomer, i.e., a single polypeptide chain, or can be a dimer, a trimer, tetramer, and/or higher order oligomer. The life-time of the oxygenated Fe²⁺ state of a heme-containing protein can be similar to that of myoglobin or can exceed it by 10%, 20%, 30%, 50%, 100% or more under conditions in which the heme-protein-containing consumable is manufactured, stored, handled or prepared for consumption. The life-time of the unoxygenated Fe²⁺ state of a heme-containing protein can be similar to that of myoglobin or can exceed it by 10%, 20%, 30%, 50%, 100% or more under conditions in which the heme-protein-containing consumable is manufactured, stored, handled or prepared for consumption.

Non-limiting examples of heme-containing polypeptides can include an androglobin, a cytoglobin, a globin E, a globin X, a globin Y, a hemoglobin, a myoglobin (e.g., bovine myoglobin), an erythrocruorin, a beta hemoglobin, an alpha hemoglobin, a protoglobin, a cyanoglobin, a cytoglobin, a histoglobin, a neuroglobins, a chlorocruorin, a truncated hemoglobin (e.g., HbN or HbO), a truncated 2/2 globin, a hemoglobin 3 (e.g., Glb3), a cytochrome, or a peroxidase.

In some embodiments, the heme containing polypeptide is a globin. A globin can be, for example, an androglobin, a cytoglobin, a globin E, a globin X, a globin Y, a hemoglobin, a myoglobin (e.g., bovine myoglobin), a leghemoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin, a truncated 2/2 globin, and a hemoglobin 3.

Heme-containing proteins that can be used as target products can be from mammals (e.g., farms animals such as cows, goats, sheep, pigs, ox, or rabbits), birds, plants, algae, fungi (e.g., yeast or filamentous fungi), ciliates, or bacteria. For example, a heme-containing protein can be from a mammal such as a farm animal (e.g., a cow, goat, sheep, pig, fish, ox, or rabbit) or a bird such as a turkey or chicken. Heme-containing proteins can be from a plant such as Nicotiana tabacum or Nicotiana sylvestris (tobacco); Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (mung beans), Lupinus albus (lupin), or Medicago sativa (alfalfa); Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley). Heme-containing proteins can be from fungi such as Saccharomyces cerevisiae, Pichia pastoris, Magnaporthe oryzae, Fusarium graminearum, Aspergillus oryzae, Trichoderma reesei, Myceliopthera thermophile, Kluyveramyces lactis, or Fusarium oxysporum. Heme-containing proteins can be isolated from bacteria such as Escherichia coli, Bacillus subtilis, Bacillus licheniformis, Bacillus megaterium, Synechocistis sp., Aquifex aeolicus, Methylacidiphilum infernorum, or thermophilic bacteria such as Thermophilus spp. The sequences and structure of numerous heme-containing proteins are known. See for example, Reedy, et al., Nucleic Acids Research, 2008, Vol. 36, Database issue D307-D313 and the Heme Protein Database available on the world wide web at http://hemeprotein.info/heme.php.

It will be appreciated that a target polypeptide can be a variant (e.g., comprise a mutation such as an amino acid substitution, e.g., a non-conservative or conservative amino acid substitution, an amino acid deletion, an amino acid insertion, or non-native sequence) relative to a corresponding naturally-occurring polypeptide. For example, a target polypeptide can lack one or more domains such as a transmembrane domain and/or the signal peptide of the corresponding naturally-occurring polypeptide. In some instances, a variant polypeptide can include at most 1, 2, 3, 4, 5, 6, 7, 8, 9, 10, 15, 20, 30, 40, or 50 mutations. In some instances, a variant polypeptide comprises at least 1, 2, 3, 4, 5, 6, 7, 8, 9, 10, 15, 20, 30, 40, or 50 or more mutations. In some instances, at least 1, 2, 3, 4, 5, 6, 7, 8, 9, 10, 20, 30, 40, or 50% of the sequence of a polypeptide of the disclosure can be mutated. In some instances, at most 1, 2, 3, 4, 5, 6, 7, 8, 9, 10, 20, 30, 40, or 50% of the sequence of a polypeptide can be mutated.

For example, the target product can be a virus or a small molecule such as an industrial small molecule such as succinic acid, fumaric acid, malic acid, FDCA (2,5 Furandicarboxylic acid), 3-HPA (3-hydroxypropionaldehyde), aspartic acid, glucaric acid, glutamic acid, itaconic acid, levulinic acid, 3-hydroxybutyrolactone, glycerol, sorbitol, xylitol, or arabitinol, or a pharmaceutical small molecule. The pharmaceutical small molecule can be a secondary metabolite from a plant biosynthesis pathway. Non-limiting examples of secondary metabolites include anthocyanins, tannins, terpenoids, alkaloids, flavonoids such as flavones or flavanoids, phenols, polyphenols, steroids such as saponins, and caffeine. The secondary metabolite can be endogenous to the transgenic plant or heterologous to the transgenic plant. For example, the secondary metabolite can be artemisinin, Paclitaxel, podophyllotoxin, or vinblastine.

Using the methods described herein, when the target product is a protein (e.g., a heme-containing polypeptide), the target product can be at least 0.01%, 0.05%, 1%, 2%, 3%, 4%, 5%, 6%, 7%, 8%, 9%, 10% or more of total protein (e.g., total soluble protein) of the transgenic seedling or plant. When the target product is a small molecule, at least 1 mg (e.g., 1.5, 2, 5, 10, 15, 20, 30, 40, 50 or more mg) of the target product can be produced per kg of transgenic plant tissue.

### Producing Transgenic Plant Cells and Plants

Transgenic plant cells and plants comprising at least one recombinant nucleic acid construct described herein can be produced using a variety of techniques. For example, Agrobacterium-mediated transformation, viral vector-mediated transformation, electroporation, or particle gun transformation can be used for introducing nucleic acids into monocotyledonous or dicotyledonous plants. See, for example, U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

The polynucleotides and vectors described herein can be used to transform a number of monocotyledonous and dicotyledonous plants including, for example, Arabidopsis, Oryza sativa (rice), Glycine max (soybean), a beet, a sugar beet, parsnip, a bean such as an adzuki, a mung, a pea, a peanut, a lentil, or a garbanzo, a leafy vegetable such as an alfalfa, an arugula, a mustard, or a Brassica, or a grass such as a barley, an oat, a wheat, a corn, a rye, triticale, or spelt.

A population of transgenic plants can be screened and/or selected for those members of the population that produce the target product. For example, a population of progeny of a single transformation event can be screened for those plants having a desired level of expression of a target product or nucleic acid encoding the target product.

Physical and biochemical methods can be used to identify expression levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or nucleic acids. Methods for performing all of the referenced techniques are known.

A plant or plant cell can be transformed by having a construct integrated into its genome, i.e., can be stably transformed. Stably transformed cells typically retain the introduced nucleic acid with each cell division. A plant or plant cell also can be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. As described above, as expression of the transgene is under control of an inducible promoter, such plants can be grown in a greenhouse or in a field without impacting the plant during normal growth, avoiding deleterious effects of the transgene during growth of the plants and removing any selective advantage to horizontal transfer of the DNA. Seeds are harvested from the transgenic plants and selectively germinated in contained systems. As used herein, a transgenic plant also refers to progeny of an initial transgenic plant provided the progeny inherits the transgene. “Progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅, and F₆ refer to subsequent generations of self- or sib-pollinated progeny of an F₁ plant.

Seeds produced by a transgenic plant can be grown and then selfed (or outcrossed and selfed) to obtain seeds homozygous for the nucleic acid construct if desired.

The invention will be further described in the following examples, which do not limit the scope of the invention described in the claims.

### EXAMPLES

### Example 1: Transformation of Rice and Arabidopsis and Identification of Transgenic Plants with an Alcohol Inducible Expression System for Soy Leghemoglobin Lbc2

The expression cassette for ethanol inducible expression of leghemoglobin was synthesized by SGI genomics in two parts. Firstly the nucleic acid encoding the soy leghemoglobin Lbc2 (Glyma20g33290.1, FIG. 2, SEQ ID NO: 1) from Glycine max was synthesized behind the alcA promoter from A. nidulans (FIG. 2, SEQ ID NO:4). A second expression cassette was synthesized wherein the alcR gene from A. nidulans (FIG. 2, SEQ ID NO:3) was placed behind the A. thaliana UBQ10 promoter. The two cassettes were assembled head to tail and then cloned into the binary vector, pPTN1138IF, which is a member of the pPZP family of binary vectors (see, for example, Hajdukiewicz et al., 1994, Plant Mol. Biol., 25:989-94).

The binary vector is introduced into rice and Arabidopsis thaliana using Agrobacterium-mediated transformation. The pPTN1138 vector carries a bar gene (Thompson et al., 1987, EMBO, 6:2519-23) under the control of the Agrobacterium tumefaciens nopaline synthase promoter (Pnos) and terminated using the 3′ UTR of the nopaline synthase gene. Therefore, selection of transformants is performed using the herbicide, Basta.

The transgenic plants are grown to maturation and the seeds are collected. The plants are monitored for healthy growth and no accumulation of leghemoglobin Lbc2 protein or mRNA is expected.

### Example 2: Transformation of Rice and Arabidopsis and Identification of Transgenic Plants with an Alcohol Inducible Expression System for Soy Leghemoglobin Lbc2 Targeted to the Protein Storage Vesicle

The expression cassette for ethanol inducible expression of leghemoglobin was synthesized by SGI genomics in two parts. The nucleic acid encoding the soy leghemoglobin Lbc2 (Glyma20g33290.1, FIG. 2, SEQ ID NO:1) from Glycine max was synthesized behind the alcA promoter from A. nidulans (FIG. 2, SEQ ID NO:4). The nucleic acid sequence encoding the conglycinin signal peptide (FIG. 2, SEQ ID NO:2) was added before the Lbc2 coding region to target leghemoglobin expression to the protein storage vacuole. A second expression cassette was synthesized wherein the alcR gene from A. nidulans (FIG. 2, SEQ ID NO:3) was placed behind the A. thaliana UBQ10 promoter. The two cassettes were assembled head to tail and then cloned into the binary vector, pPTN1138IF, which is a member of the pPZP family of binary vectors (see, for example, Hajdukiewicz et al., 1994, supra). The soybean sequences were placed under control of the 35S Cauliflower Mosaic Virus (CaMV) promoter with a duplicated enhancer (Benfey & Chua, 1990, Science, 250:959-66) and terminated by its 3′ UTR.

The binary vector is introduced into rice and A. thaliana using Agrobacterium-mediated transformation and transformants are selected using Basta.

The transgenic plants are grown to maturation and the seeds are collected. The plants are monitored for healthy growth and no accumulation of leghemoglobin Lbc2 protein or mRNA is expected.

### Example 3: Expression of Leghemoglobin

To germinate the transgenic seeds, the seeds are put in a container and 2-3 times as much cool water (60-70° F.) is added. The seeds are mixed to assure even water contact for all and the seeds are soaked for 8-12 hours. After soaking, the water is drained from the seeds, and the seeds are thoroughly rinsed with cool water (60-70° F.) and then drained again. Between rinses, the seeds are set out of direct sunlight and at room temperature (70° F. is optimal). The seeds are rinsed and drained again in 8-12 hours, and repeated one to three more times. Once a very short root is observed the seed is ready for planting.

After a final rinse, the seeds are spread evenly on thoroughly moistened soil, coconut coir, vermiculite or other medium. The planted seeds are covered to keep light out and moisture in, and placed in a low-light, room temperature (˜70° F.) location. The seeds are watered lightly once or twice a day to keep the sprouts moist until their roots bury themselves in the soil/medium.

When the seedlings reaches 1 inch tall, the cover is removed, and ethanol is added to the moisture surrounding the seeds to 2% final concentration. After twelve to 72 hours of induction, the leghemoglobin concentration has reached the maximum level.

### Example 4: Isolation of Leghemoglobin

One kg of seedlings are macerated in a VITA-PREP® 3 blender (Vitamix Corp., Cleveland, Ohio) in a ratio of 1:1 (w/w) with potassium phosphate buffer (pH 7.4). The extraction is performed for 3 minutes at the highest setting (3 HP motor) maintaining the temperature at less than 30° C. at all times. The pH is adjusted to 7.4 post-grinding, using a 10 M NaOH solution. The homogenate is centrifuged at 3500 g for 5 minutes using a bench top centrifuge (Allegra X15R, SX4750 rotor; Beckman Coulter, Inc., Pasadena, Calif.). The pellet is discarded and the supernatant (about 1.6 L) is collected separately. The soluble protein fraction is then microfiltered using a 0.2 μm modified polyethersulfone (mPES) membrane in a hollow fiber format (KROSFLO® K02E20U-05N; Spectrum Laboratories, Inc., Rancho Dominguez, Calif.). The filtrate from this step (about 3 L) is concentrated using a 5 kDa mPES membrane (MiniKros N02E070-05N; Spectrum Laboratories, Inc.) to about 0.1 L. The partially purified leghemoglobin solution can be further purified using Q Fast Flow anion exchange resin (GE Lifesciences). The final leghemoglobin product is concentrated using 3 kDa ultrafiltration and frozen at −20° C.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of recombinantly producing a member of the globin family in a contained system, the method comprising:
a) germinating a plurality of transgenic seeds in the contained system to produce a plurality of seedlings or plants, wherein each transgenic seed comprises a nucleic acid construct, the nucleic acid construct comprising an inducible promoter operably linked to (i) a nucleic acid encoding the member of the globin family or (ii) a nucleic acid encoding a polypeptide in a biosynthesis pathway of heme;
b) contacting the seedlings or plants with an inducer for the inducible promoter; and
c) isolating the member of the globin family from the plurality of seedlings or plants.

2. A method of recombinantly producing a member of the globin family in a contained system, the method comprising:
a) germinating a plurality of transgenic seeds in the contained system in the presence of an inducer to produce a plurality of seedlings, wherein each transgenic seed comprises a nucleic acid construct, the nucleic acid construct comprising an inducible promoter operably linked to (i) a nucleic acid encoding the member of the globin family or (ii) a nucleic acid encoding a polypeptide in a biosynthesis pathway of heme; and
b) isolating the target product from the plurality of seedlings.

3. A method of recombinantly producing a member of the globin family in a contained system, the method comprising:
a) germinating a plurality of transgenic seeds in the contained system to produce a plurality of seedlings, wherein each transgenic seed comprises a nucleic acid construct, the nucleic acid construct comprising a germination specific promoter operably linked to (i) a nucleic acid encoding the member of the globin family or (ii) a nucleic acid encoding a polypeptide in a biosynthesis pathway of heme; and
b) isolating the target product from the plurality of seedlings.

4. The method of claim 2, wherein the member of the globin family is at least 0.01% of the total protein from the seedling or plant.

5. The method of claim 4, wherein the member of the globin family is at least 0.01% of the total soluble protein from the seedling or plant.

6. The method of claim 2, wherein the isolating step comprises processing the plurality of seedlings or plants and purifying the member of the globin family from the processed seedlings or plants.

7. (canceled)

8. (canceled)

9. The method of claim 2, wherein the nucleic acid construct further encodes a targeting sequence.

10. The method of claim 9, wherein the targeting sequence is a root targeting sequence.

11. The method of claim 9, wherein the targeting sequence is a hypocotyl targeting sequence.

12. The method of claim 9, wherein the targeting sequence is a vacuole targeting sequence.

13. The method of claim 2, wherein the member of the globin family is expressed throughout the seedling.

14.-33. (canceled)

34. The method of claim 2, wherein the contained system is a malting system.

35. (canceled)

36. The method of claim 34, wherein the plurality of seeds is germinated by (i) steeping the plurality of transgenic seeds in the presence of water and optional inducer until the moisture content of the plurality of transgenic seeds ranges from about 30% to about 50% and (ii) ventilating the steeped plurality of seeds at a temperature from about 15° C. to about 25° C. in the presence of the inducer; and the member of the globin family is isolated from the ventilated seedling.

37. The method of claim 2, wherein the member of the globin family is thermostable.

38. The method of claim 37, wherein the plurality of seedlings is kilned before isolation.

39. The method of claim 2, wherein the contained system is a hydroponic system.

40. The method of claim 2, wherein the plant is from 1 to 50 days old when contacted with the inducer.

41. The method of claim 2, wherein the member of the globin family is isolated from the plant 6 to 100 hours after being contacted with the inducer.

42. The method of claim 2, wherein the member of the globin family is leghemoglobin (lbc2), a non-symbiotic hemoglobin, an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin, a truncated 2/2 globin, a hemoglobin 3, a cytochrome, or a peroxidase.

43. The method of claim 2, wherein the transgenic seeds are soybean seeds.

44. (canceled)

45. The method of claim 2, wherein the transgenic seeds are selected from the group consisting of a beet, a sugar beet, parsnip, a bean, a leafy vegetable, and a grass seed.

46. (canceled)

47. (canceled)

48. The method of claim 45, wherein the grass is a barley, a wheat, a corn, a rye, an oat, triticale, or spelt.


SEQUENCE LISTING (5 sequences)

SEQ ID NO: 1 - DNA, 438 bp, Glycine max
atgggtgctttcactgagaagcaagaggctttggtgagtagctcattcgaagcattcaaggcaaacattcctcaatacagcgttgtgttctacacttcgatactggagaaagcacccgcagcaaaggacttgttctcgtttctatctaatggagtagatcctagtaatcctaagctcacgggccatgctgaaaagctttttggattggtgcgtgactcagctggtcaacttaaagcaaatggaacagtagtggctgatgccgcacttggttctatccatgcccaaaaagcaatcactgatcctcagttcgtggtggttaaagaagcactgctgaaaacaataaaggaggcagttggggacaaatggagtgatgaattgagcagtgcttgggaagtagcctatgatgaattggcagcagctattaagaaggcattttag

SEQ ID NO: 2 - DNA, 186 bp, Artificial
  synthetic oligonucleotide
atgatgagagcgcggttcccattactgttgctgggagttgttttcctagcatcagtttctgtctcatttggcattgcgtattgggaaaagcagaaccccagtcacaacaagtgcctccgaagttgcaatagcgagaaagactcctacaggaaccaagcatgccacgctcgttgcaacctccttaag

SEQ ID NO: 3 - DNA, 2466 bp, Aspergillus nidulans
atggcagatacgcgccgacgccagaatcatagctgcgatccctgtcgcaagggcaagcgacgctgtgatgccccggaaaatagaaacgaggccaatgaaaacggctgggtttcgtgttcaaattgcaagcgttggaacaaggattgtaccttcaattggctctcatcccaacgctccaaggcaaaaggggctgcacctagagcgagaacaaagaaagccaggaccgcaacaaccaccagtgaaccatcaacttcagctgcaacaatccctacaccggaaagtgacaatcacgatgcgcctccagtcataaactctcacgacgcgctcccgagctggactcaggggctactctcccaccccggcgaccttttcgatttcagccactctgctattcccgcaaatgcagaagatgcggccaacgtgcagtcagacgcaccttttccgtgggatctagccatccccggtgatttcagcatgggccaacagctcgagaaacctctcagtccgctcagttttcaagcagtccttcttccgccccatagcccgaacacggatgacctcattcgcgagctggaagagcagactacggatccggactcggttaccgatactaatagtgtacaacaggtcgctcaagatggatcgctatggtctgatcggcagtcgccgctactgcctgagaacagtctgtgcatggcctcagacagcacagcacggcgatatgcccgttccacaatgacgaagaatctgatgcgaatctaccacgatagtatggagaatgcactgtcctgctggctgacagagcacaattgtccatactccgaccagatcagctacctgccgcccaagcagcgggcggaatggggcccgaactggtcaaacaggatgtgcatccgggtgtgccggctagatcgcgtatctacctcattacgcgggcgcgccctgagtgcggaagaggacaaagccgcagcccgagccctgcatctggcgatcgtagcttttgcgtcgcaatggacgcagcatgcgcagaggggggctgggctaaatgttcctgcagacatagccgccgatgagaggtccatccggaggaacgcctggaatgaagcacgccatgccttgcagcacacgacagggattccatcattccgggttatatttgcgaatatcatcttttctctcacgcagagtgtgctggatgatgatgagcagcacggtatgggtgcacgtctagacaagctactcgaaaatgacggtgcgcccgtgttcctggaaaccgcgaaccgtcagctttatacattccgacataagtttgcacgaatgcaacgccgcggtaaggctttcaacaggctcccgggaggatctgtcgcatcgacattcgccggtattttcgagacaccgacgccgtcgtctgaaagcccacagcttgacccggttgtggccagtgaggagcatcgcagtacattaagccttatgttctggctagggatcatgttcgatacactaagcgctgcaatgtaccagcgaccactcgtggtgtcagatgaggatagccagatatcatcggcatctccaccaaggcgcggcgctgaaacgccgatcaacctagactgctgggagcccccgagacaggtcccgagcaatcaagaaaagagcgacgtatggggcgacctcttcctccgcacctcggactctctcccagatcacgaatcccacacacaaatctctcagccagcggctcgatggccctgcacctacgaacaggccgccgccgctctctcctctgcaacgcccgtcaaagtcctcctctaccgccgcgtcacgcagctccaaaccctcctctatcgcggcgccagccctgcccgccttgaagcggccatccagagaacgctctacgtttataatcactggacagcgaagtaccaaccatttatgcaggactgcgttgctaaccacgagctcctcccttcgcgcatccagtcttggtacgtcattctagacggtcactggcatctagccgcgatgttgctagcggacgttttggagagcatcgaccgcgattcgtactctgatatcaaccacatcgaccttgtaacaaagctaaggctcgataatgcactagcagttagtgcccttgcgcgctcttcactccgaggccaggagctggacccgggcaaagcatctccgatgtatcgccatttccatgattctctgaccgaggtggcattcctggtagaaccgtggaccgtcgttcttattcactcgtttgccaaagctgcgtatatcttgctggactgtttagatctggacggccaaggaaatgcactagcggggtacctgcagctgcggcaaaattgcaactactgcattcgggcgctgcaatttctgggcaggaagtcggatatggcggcgctggttgcgaaggatttagagagaggtttgaatgggaaagttgacagctttttgtag

SEQ ID NO: 4 - DNA, 257 bp, Aspergillus nidulans
ccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaaca

SEQ ID NO: 5 - protein, 4 aa, Artificial
  synthetic peptide
NPIR